(19) United States
(12) Patent Application Publication
(10) Pub. No.: US 2025/0171726 A1
(43) Pub. Date: May 29, 2025

(54) OPTIMIZED HAIRY ROOTS BIOREACTOR

(71) Applicant: SAMABRIVA, Amiens (FR)

(72) Inventors: Florian CARDON, Amiens (FR); Camille LEMASSON, Amiens (FR); Marina GUILLET, Amiens (FR)

(73) Assignee: SAMABRIVA, Amiens (FR)

(21) Appl. No.: 18/841,425

(22) PCT Filed: Mar. 31, 2023
(86) PCT No.: PCT/EP2023/058535

§ 371 Date: Aug. 26, 2024

(30) Foreign Application Priority Data
Apr. 1, 2022 (EP) 22305436.2

(51) Int. Cl. C12M 1/00; C12M 1/12

(52) U.S. Cl. CPC C12M 29/08; C12M 23/06; C12M 23/26; C12M 23/28; C12M 23/34

(57) ABSTRACT

A bioreactor for the production of biomass configured to contain at least 15 liters, with an external support structure displaying a general annular shape, an internal tank being configured to be secured inside and supported by the external support structure. The internal space of the internal tank is free of any internal structure. The bioreactor includes a sparger located at a closed extremity of the internal tank. The sparger is configured to generate bubbles displaying a diameter of at least 5 millimeters. The sparger and the internal tank are arranged in a way that the generated bubbles form one bubble column once the internal tank is filled with liquid and the sparger is fed with gas, the bubble column enabling the liquid to be put in motion.

### FIELD OF INVENTION

The present invention relates to growing biomass, in particular with the aim of producing molecules of interest (like for example recombinant proteins and/or secondary metabolites), in an optimized bioreactor.

### BACKGROUND OF INVENTION

Hairy roots from plants have been widely studied and used for the production of specialized/secondary metabolites of industrial and pharmaceutical interest. Since the 1990s, the production of recombinant proteins has been considered as another promising application of hairy root cultures. This system presents numerous similarities with the one used to produce recombinant proteins from mammalian cell lines (such as, e.g., Chinese hamster ovary (CHO) cells, human cell lines, bacteria), among which the fact that the whole process is maintained under sterile conditions in a confined environment.

This plant-based technology offers relevant and advantageous differences compared to the mammalian expression systems. The selected hairy root clones are easily grown in tailor-made optimal conditions in a simple culture medium which has a better safety profile than culture media containing human- or animal-derived constituents. This process allows the large-scale production of compounds (often referred to as molecules of interest) such as recombinant proteins or secondary metabolites in an axenic environment in bioreactors. Moreover, contrary to the common mammalian cells-based expression systems, the hairy-root expression system allows a straightforward recovery of the molecules of interest that does not require an additional extraction phase.

However, as in every production method, it is important to obtain the highest possible amount of molecules of interest (like proteins or metabolites) in a same bioreactor in order to preserve an optimal reproducibility and decrease the number of bioreactors used in parallel in an attempt to reduce the carbon footprint.

### SUMMARY

This invention thus relates to a bioreactor for the production of biomass, the bioreactor being configured to contain at least 15 liters of a liquid comprising:
an external support structure displaying a general annular shape extending along a revolution axis,
a generally cylindrical shaped internal tank made of flexible material, said internal tank being configured to be secured inside and supported by the external support structure, the internal tank further displaying a closed extremity,
wherein the internal space of the internal tank is free of any internal structure,
wherein the bioreactor further comprises a sparger located at the closed extremity of the internal tank,
wherein the sparger is configured to generate bubbles displaying a diameter of at least 5 millimeters,
wherein the sparger and the internal tank are arranged in a way that the generated bubbles form one bubble column once the internal tank is filled with liquid and the sparger is fed with gas, said bubble column enabling the liquid to be put in motion.

Thus, this solution achieves the above objective. In particular, it makes it possible to obtain a significant improvement in the productivity of molecules of interest while maintaining a high biomass growth by using bubbles of at least 5 mm of diameter.

The bioreactor according to the invention may include one or more of the following characteristics, taken in isolation from one another or in combination with one another:
the sparger may be arranged and secured in the center of the closed extremity, centered with regard to the revolution axis X,
the sparger may comprise at least one radial aperture located along a revolution axis A of the sparger, said aperture being designed to generate a column of bubbles,
the sparger may comprise two alignments of at least one radial aperture located along a revolution axis A of the sparger, the two alignments being on opposite sides of the sparger, said apertures being designed to generate at least one column of bubbles,
the generated bubbles may have a diameter D ranging from 5 to 17 mm,
the bioreactor may be configured to contain at least 100 liters of liquid,
the bioreactor may be configured to contain at least 350 liters of liquid,
the bioreactor may be configured to contain at least 1000 liters of liquid,
the internal tank may be disposable,
the bioreactor may further comprise a control system,
the closed extremity may display a conical shape centered around the revolution axis,
the sparger may be designed to cooperate with the conical shape of the closed extremity,
the bioreactor may be an air-lift system in which the liquid is solely put in motion by the generated air bubbles.

A further object of the present invention is a system for the production of biomass, said system comprising a bioreactor according to any one of the here-above listed technical features and a gas injection device, the gas injection device being put in fluidic communication with the sparger.

A third object of the present invention is a method for growing biomass in a culture medium, said culture medium being put in motion by a bioreactor according to any one of the here-above listed technical features, said method comprising the following steps:
filling the internal tank with culture medium,
inserting the biomass aimed at being grown in the culture medium,
feeding the sparger with gas in order to generate the bubble column aimed at putting the culture medium into motion.

### BRIEF DESCRIPTION OF THE DRAWINGS

The invention will be better understood, and other aims, details, characteristics and advantages thereof will emerge more clearly on reading the detailed explanatory description which follows, including embodiments of the invention given by way of illustration and purely illustrative and non-limiting examples, with reference to the accompanying drawings:

FIG. 1 is a schematic view of the bioreactor according to the invention,

FIG. 2 is a perspective view of the bioreactor according to the invention filled with liquid,

FIG. 3 is a schematic view of the internal tank of the bioreactor according to the present invention filled with gas and liquid,

FIG. 4 is a schematic longitudinal section of a first embodiment of the internal tank of the bioreactor according to the present invention,

FIG. 5 is a schematic longitudinal section of a second embodiment of the internal tank of the bioreactor according to the present invention,

FIG. 6 is a transparent perspective view from a sparger according to the present invention,

FIG. 7 is a view from above from the sparger according to FIG. 6,

FIGS. 8a and 8b are longitudinal section views of the sparger according to FIG. 6,

FIG. 9 are charts comparing bubble surfaces in function of water heights using different sorts of spargers,

FIG. 10 are charts comparing biomass concentration and protein productivity using respectively a wave bioreactor and a large bubble sparger according to the present invention,

FIG. 11 are charts comparing biomass concentration and protein productivity using respectively a microbubble sparger and a large bubble sparger according to the present invention.

### DETAILED DESCRIPTION

The invention thus relates to a bioreactor 10 for the production of biomass, such as hairy roots, in particular with the aim of producing molecules of interest. The bioreactor is a column like reactor extending along a longitudinal axis X with a bottom extremity 10A and an upper extremity 10B.

In the present invention, the terminology “biomass” refers to any living organisms that may be cultured (or grown) in vitro. In some embodiments, the biomass refers to a plant (i.e., plant biomass), in particular to plant roots (i.e., root biomass). Example of plant roots that may be grown in the bioreactor include hairy roots, rhizocals, and adventitious roots. Thus, in some embodiments, the biomass is adventitious roots, hairy roots, rhizocals, or any combination thereof. In some embodiments, the biomass is hairy roots (i.e., hairy root biomass).

The terminology “adventitious roots” refers to root emergences which appear during the physiological development of the plant and hence occurs naturally. The terminology “hairy roots” refers to root emergences which appear after the infection of a plant by Rhizobium rhizogenes (previously referred to as Agrobacterium rhizogenes) bacteria or by Rhizobium radiobacter (also known as Agrobacterium Tumefaciens) bacteria harboring rol genes for example. The terminology “rhizocal” or “rhizocallus” refers to a conic-shaped structure connected to the roots (in particular to the hairy roots), also termed lateral root emergence, which develops alongside of the roots in a solidarized way. In practice, rhizocals (or “rhizocalli”) may be induced in a culture of roots by the addition of auxin in the culture medium.

The terminology “molecule of interest” refers to molecules (or compounds), such as recombinant proteins and/or secondary metabolites, which may be produced (and likely secreted) by the biomass, in particular by roots such as hairy roots.

The bioreactor 10, more precisely the internal tank 14 of the bioreactor 10, is configured to contain at least 15 liters of a liquid. In the present invention, the terminology “a liquid” or “liquid” refers to at least one liquid. In the other words, in the present invention, the terminology “a liquid” or “liquid” may include one or several liquids. In some embodiment, the liquid is a culture medium.

Roots, and in particular hairy roots are commonly cultivated (or grown) on solid medium or in liquid medium. Hairy roots may be cultivated in a standard culture medium (such as Gamborg B5, MS, etc.) or using an optimized culture medium where each nutrient concentration is specifically defined to allow better growth and/or molecule productivity. In a preferred embodiment, the internal tank 14 of the bioreactor 10 is configured to contain a volume ranging from 200 to 350 L of liquid. In some embodiments, the internal tank 14 of the bioreactor 10 can contain up to 1000 L of liquid, and even up to 5000 L. The internal tank 14 is classically put together by welding several pieces together.

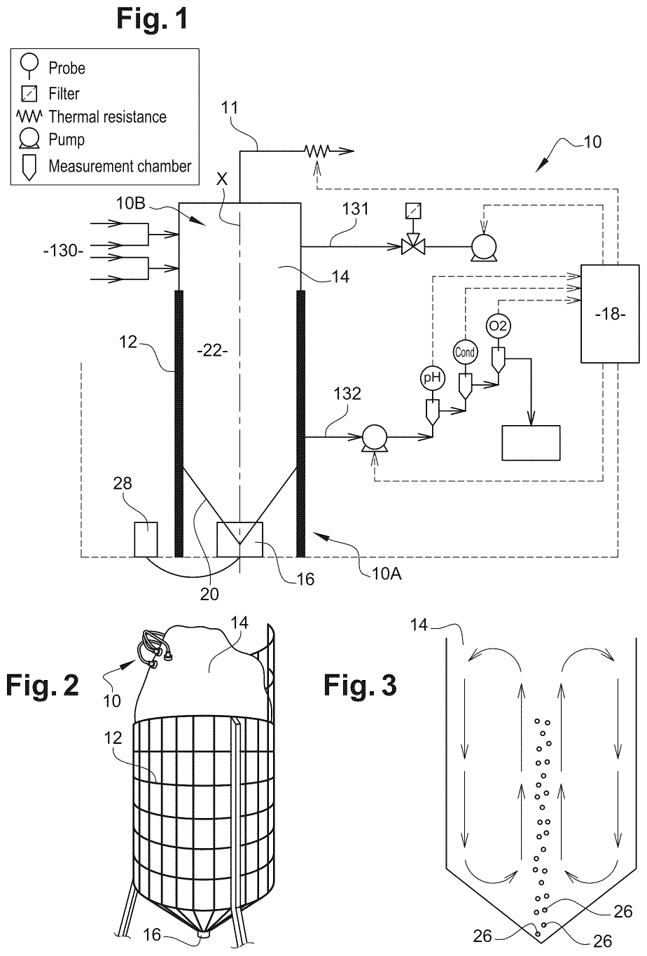

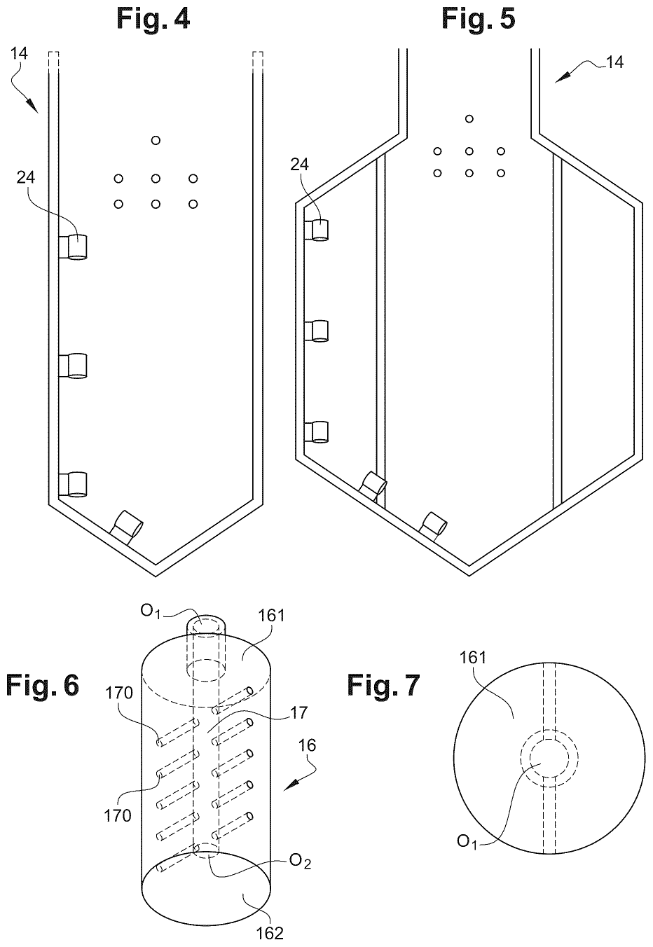

In these regards, as can be seen on the embodiment depicted on FIG. 1, the bioreactor 10 comprises:
an external support structure 12 displaying a general annular shape extending along a revolution axis X, also being the longitudinal axis X,
a generally cylindrical shaped internal tank 14, being configured to be secured inside and supported by the external support structure 12,
a sparger 16 located at the closed extremity 20 of the internal tank 14,
a control system 18 comprising hardware and software.

The bioreactor 10 according to the present invention might be part of a system for the production of biomass, said system comprising a bioreactor 10 and a gas injection device put in fluidic communication with the sparger 16.

The external support structure 12 is designed to hold the internal tank 14 as a sheath, as can be seen on FIG. 2. In some embodiment, as can be seen on FIG. 1, the external support structure 12 can be made of a stainless-steel mesh. The external support structure 12 may thus withstand the pressure applied by the liquid inside the internal tank 14 and avoid that this pressure is withstood solely by the plastic and welding sites of the internal tank 14. This allows to avoid any leaks at the different welding sites.

As can also be seen on FIG. 2, the internal tank 14 is made of flexible material, as for example, a polypropylene/polyamide double-layer plastic film, displaying a thickness of 115 μm. In any case, the material should be a gas and liquid barrier plastic film.

In a preferred embodiment, the internal tank 14 is disposable. This makes it easier to grow the biomass in sterile conditions.

More precisely, the internal tank 14 is made of five strips welded together. This makes it possible, once the internal tank 14 is filled with liquid, to form a reservoir much closer to a cylinder than if the internal tank 14 had been made from one or two strips. This allows the pressure forces exerted by the liquid filling the internal tank 14 to be distributed more evenly over the entire internal surface of the internal tank and along the different welding sites.

The internal tank 14 displays at least one closed extremity 20. In some embodiments, said closed extremity 20 can display a conical shape, the cone being centered around the revolution axis X. The conical shape of the closed extremity 20 of the internal tank 14 of the bioreactor 10 makes it possible both to reduce dead volumes and also to facilitate recirculation of the liquid. This closed extremity 20 is configured to be the bottom extremity 10A of the bioreactor 10. In some embodiments, in which the biomass has to been grown in a sterile environment, the upper extremity of the internal tank 14 (being the upper extremity 10B of the bioreactor 10) is also closed and comprises an air outlet 11. Said upper extremity may further comprise several inlet conducts 130 to add liquid and nutrients, one first outlet conduct 131 to collect sample and one second outlet conduct 132 to collect trash.

Depending on the embodiments, the internal tank 14 presents a height ranging from 1000 to 5000 mm and a width ranging from 300 to 1000 mm.

The internal volume 22 of the internal tank 14 is free of any internal structure, in particular any internal support structure. This means that the internal volume 22 of the internal tank 14 is one single continuous space: no internal support structure like a transverse or crossing conduct, an internal strand or an internal wall is to be found inside the internal tank 14 in order to separate this unique internal volume 22 into several internal spaces.

In some embodiments, the internal tank 14 comprises at least one loop 24 secured to its wall. The at least one loop 24 is configured to hold at least one fluidic conduct and to maintain said fluidic conduct in contact with the wall. The fluidic conduct might be used to transport some water or fresh culture medium. In some embodiments, the loop 24 is secured on the external face of the wall of the internal tank 14 and in some alternative embodiments, the loop 24 is secured on the internal face of the wall of the internal tank 14. However, even if the loop 24 is secured on the internal face of the wall, said loop and the associated fluidic conduct being maintained in contact with the wall, they do not separate the unique internal volume 22 into several internal spaces. Neither the loop 24 nor the fluidic conducts can be considered as internal support structures.

The design of the internal tank 14 of the bioreactor 10 includes different factors among which the geometric ratio (r_{G}), which is defined, in the present application, as being the ratio between the diameter d and the useful height H (H being the height of the liquid column inside the internal tank 14) of the internal tank 14:

$${r}_{G} ( - ) = \frac{d ( m )}{H ( m )}$$

This ratio defines the size of the internal tank 14 and thus the space available for the biomass (for example hairy roots) to grow. Different ratios can be used regarding the present invention depending on the volume of the internal tank 14. For a 25 L scale, r_{G} ranges between 0.1 and 0.2, for a 200 L scale, r_{G} ranges between 0.3 and 0.5 and for a 350 L scale, r_{G} ranges between 0.4 and 0.6. Empirical knowledge indicates that these ratios r_{G} intervals have no impact on the hairy roots growth.

The sparger 16 and the internal tank 14 are arranged in a way that the generated bubbles 26 form a bubble column once the internal tank 14 is filled with liquid and the sparger 16 is fed with gas. In a preferred embodiment, it is one single bubble column. The bubble 26 column enables the liquid to be put in motion.

As can be seen on FIG. 6, the sparger 16 preferably displays a general cylindrical shape extending along a revolution axis A with a diameter ranging from 10 to 20 mm and a length ranging from 30 mm to 100 mm. When the sparger 16 is inserted in the internal tank 14, the revolution axis A of the sparger 16 aligns with the revolution axis X of the internal tank 14. As can be grabbed on FIGS. 6, 7 and 8a, the sparger 16 comprises an upstream extremity 161 and a downstream extremity 162. The upstream extremity 161 comprises an opening O which enables the sparger to be fed with gas. The opening O extends, along the revolution axis A, outwards the upstream extremity 161. This shape enables a better gas distribution inside the sparger 16 and avoids any dead volumes.

The walls of the sparger can be made of inox, metal or plastic, for example.

An internal conduct 17 presenting a diameter ranging from 2 to 5 mm extends downstream from the opening O and enables gas to enter the sparger 16 through the opening O. The sparger 16 further comprises, on its external wall, at least one radial aperture 170 put radially in fluidic communication with the internal conduct 17. In some embodiments, the sparger 16 comprises several radial apertures 170. The opening O is thus put in fluidic communication with each radial aperture 170 by means of the internal conduct 17.

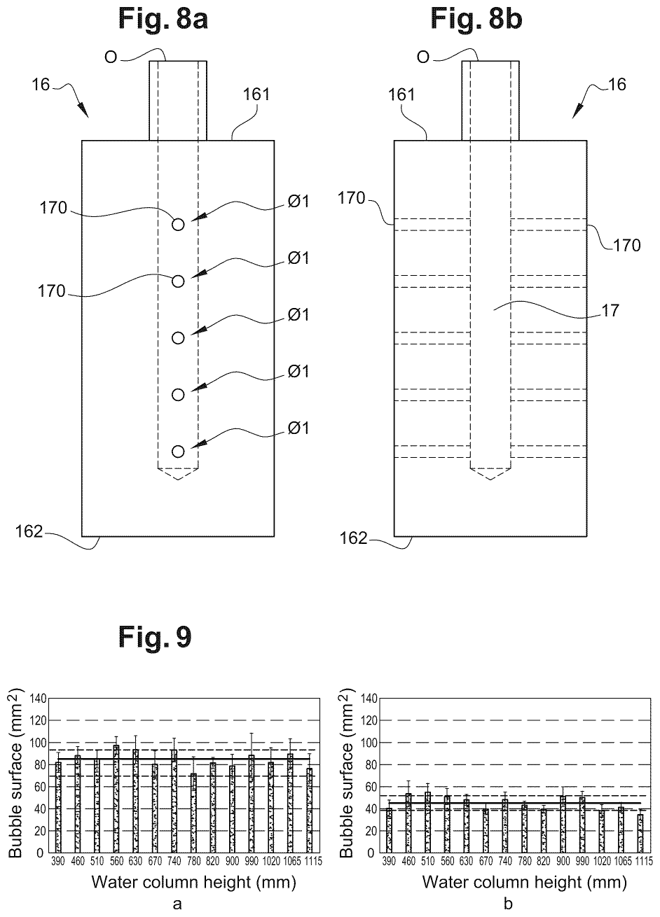

The at least radial aperture 170 presents a diameter ranging from 0.5 to 1.5 mm, preferably 1 mm. In the embodiment depicted on FIGS. 6, 8a and 8b, the sparger 16 displays ten radial apertures 170, five on each side of the sparger 16. On each side, all five radial apertures 170 are aligned along the revolution axis A. Once the sparger 16 is fed with gas, each radial aperture 170 enables to produce a series of gas bubbles 26 presenting a diameter D ranging from 5 to 17 mm.

The sparger 16 is therefore preferably arranged and secured in the center of the closed extremity 20 of the internal tank 14. The sparger 16 is centered with regard to the revolution axis X, thus enabling the formation of a central air column.

This makes the bioreactor 10 according to the present invention of the “bubble column” type, meaning that the agitation of the liquid is generated by the introduction of gas at the bottom of the bioreactor. Moreover, in the bioreactor 10 of the present invention being an air-lift system, the air flow of the bubble column directly influences the agitation and the type of flow in the internal tank 14 of the bioreactor 10. The higher the air of the bubble column flows, the more the liquid flow is increased. Some examples of experimental results will be discussed further below.

In order to feed the sparger 16 with gas, preferably air, the sparger 16 is put in fluidic communication with a gas injection device 28, preferably an air injection device. The gas injection device 28 is designed to generate a gas flow rate (preferably air flow rate) ranging from 0.5 L/min to 10 L/min, preferably 1 L/min.

When the sparger is fed with gas while the internal tank 14 is filled with liquid, the internal volume 22 of the internal tank 14 is separated into a gassed and an ungassed region. The bubbles generate a vertically circulating flow between the gassed and ungassed regions, as can be seen on FIG. 3. The internal volume 22 is solely separated by the flowing air of the generated bubble column(s).

The control system 18 allows the acquisition and/or the control of several monitoring signals inside the liquid like for example pH, conductivity, dissolved oxygen. The control system comprises for example a control box like BioFlo® 120 Eppendorf or Biostat® B Sartorius. This control system 18 thus allows a precise monitoring of the growing process of the biomass.

The bioreactor 10 according to the present invention enables the implementation of a method for growing biomass in a culture medium, said culture medium being put in motion by air bubbles 26, said method comprising the following steps:
filling the internal tank 14 with culture medium,
inserting the biomass aimed at being grown or cultivated in the culture medium,
turning the sparger 16 on in order to generate the bubble 26 column aimed at putting the culture medium into motion.

### Examples

The objective of the herein presented experiments was to compare two bioreactors with bubbling systems based on two sparger technologies. For that, the bubble size distribution in function of the water column height was measured by picture acquisition and image analysis. Furthermore, the effect of the bubble size on the growth of the biomass and on the production of a recombinant protein was assessed.

### Materials and Methods

### Bioreactor and Aeration Conditions

A single-use bioreactor containing 25 L of distilled water was used for the bubble size measurement acquisition. In order to obtain clear and workable pictures, the air flow rate was maintained at 1 L/min during all the experiment and for both spargers. The spargers were placed at the bottom of the bioreactor.

### Bubble Pictures Acquisition

Pictures were taken using a camera (Canon®) mounted on a tripod with adjustable height. The distance between the camera and the bioreactor was set at 185 mm during all the experiment in order to have exactly the same capture conditions. Pictures were taken from the bottom of the bioreactor to the top with regular height steps.

### Image Analysis

Image analysis was done using the software ImageJ. The “Ellipse” tool of the software was used to encircle each bubble on the pictures acquired. The “Measurement Analysis” tool was then used to calculate the area of the encircled bubbles. As this data was given by the software in pixel units, a calibration was needed to know the equivalence between pixels and distance in mm. For that, a control consisting in a sphere of 50 mm diameter was used for pixels calibration. A picture of the sphere was acquired in exactly the same conditions as the bubbles and the picture analysis has allowed to establish the equivalence between millimeters and pixels. This calibration was used to calculate the bubble area in square millimeters. Bubble areas were then averaged for each height step in order to represent the distribution of the average area in function of the height of the bioreactor, for both spargers.

### Biomass Concentration Measurement

Hairy roots of Brassica rapa rapa producing the protein IDUA (alpha-L-iduronidase) were cultivated in a bioreactor in standard conditions. At the end of the hairy root culture, the roots were collected, washed in protein recovery solution, rinsed with distilled water and dried at 70° C. during 48 h. Dry weight (in g_{DW}) was then measured and reported to the culture volume (in L) to calculate the biomass concentration (in g_{DW}/L).

### IDUA Activity and Productivity

To assess the amount of IDUA protein produced by the hairy root culture, the enzymatic activity of the IDUA protein was measured in the collected protein recovery solution using a fluorimetric enzymatic assay based on the fluorogenic substrate sodium 4-methylumbelliferyl-α-L-Iduronide (4MU-I; Santa Cruz Biotechnology). The 4MUI substrate was diluted to a working solution of 400 μM 4MU-I with the reaction buffer 0.4 M sodium formate, pH 3.5. Twenty-five μL of sample to analyze were added to 25 μL of 400 μM 4MU-I substrate. The mixture was incubated at 37° C. for 30 min and 200 μL glycine carbonate buffer (pH 9.8) was added to quench the reaction. 4-Methylumbelliferone (4MU) (Sigma) was used to prepare the standard calibration curve. Fluorescence was measured using a plate reader (TECAN Infinite M1000™ Männedorf, Switzerland) with excitation at 355 nm and emission at 460 nm. IDUA activity in measured in enzymatic unit per liter of sample (U/L). IDUA productivity (in mU/g_{DW}/d) was then calculated by dividing the IDUA units (in U) by the dry weight (g_{DW}) and by the production duration (in days).

### Results and Discussion

### FIG. 9—Bubble Size Measurement

FIG. 9a expresses the bubble surface (in mm²) in function of the water column height (in mm) when using a large bubble sparger according to the present invention. The average surface is 85.2 mm² (IC₉₅=11.41) corresponding to an average diameter of about 5.2 mm. FIG. 9b expresses the bubble surface (in mm²) in function of the water column height (in mm) when using a microbubble sparger. The average surface is 42.26 mm² (IC₉₅=6.3) corresponding to an average diameter of about 3.65 mm.

The conclusion that can be drawn from those results is that the generated bubbles are twice smaller when using a microbubble sparger than when using a large bubble sparger like in the present invention.

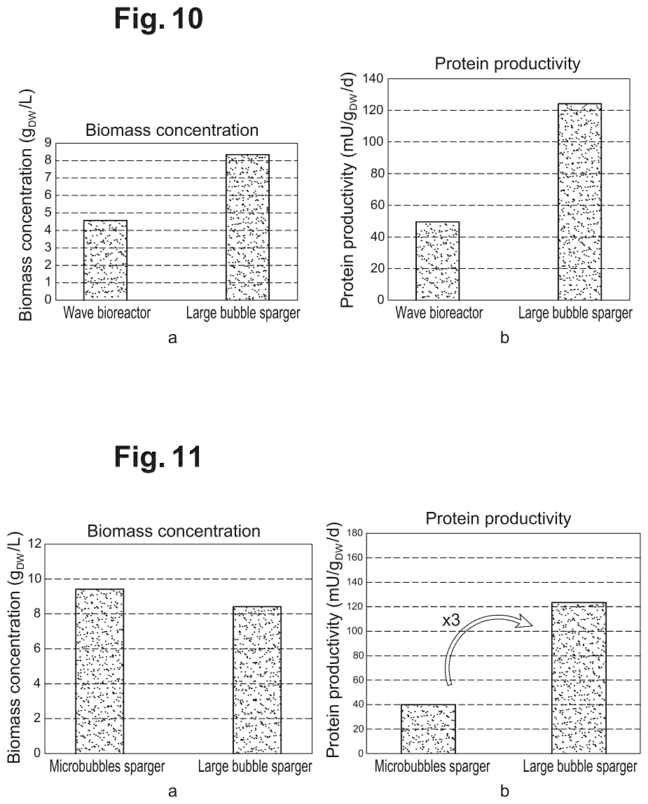

FIGS. 10a and 10b—Comparison with a Wave Bioreactor

FIG. 10a shows the difference of biomass concentration in g_{DW}/L and the protein productivity in mU/g_{DW}/d obtained after 39 days of culture while using (on the left) a commercially available wave bioreactor and (on the right) a bioreactor with a large bubble sparger according to the present invention. A wave bioreactor does not oxygenate the culture medium by means of bubbles, the oxygenation is solely provided by agitation.

It appears quite clearly that the biomass concentration obtained after 39 days of culture is about two times greater when using a large bubble sparger according to the present invention than when using a commercially available wave bioreactor.

It also appears quite clearly that the amount of protein produced after 39 days of culture is about three times greater when using a large bubble sparger according to the present invention than when using a wave bioreactor.

FIGS. 11a and 11b—Comparison with a Microbubble Sparger

FIG. 11a shows the biomass concentration in g_{DW}/L obtained after 39 days of culture while using (on the left) a bioreactor with a microbubble sparger and (on the right) a bioreactor with a large bubble sparger according to the present invention.

No significant impact on biomass production can be detected.

FIG. 11b shows the difference in the amount of protein produced after 39 days of culture, expressed in mU/g_{DW}/d when using (on the left) a bioreactor with a microbubble sparger and (on the right) a bioreactor with a large bubble sparger according to the present invention.

Despite the similar biomass concentration observed in FIG. 11a, it appears quite clearly that using a bioreactor with a large bubble sparger according to the present invention leads to a protein production about three times greater than when using a microbubble bioreactor.

### CONCLUSION

Therefore, using a bioreactor with a large bubble sparger like in the present invention allows to significantly increase the protein production by a hairy root culture, notably in comparison to the protein production by a hairy root culture in a bioreactor with a microbubble sparger or in a wave bioreactor.

1-15. (canceled)

16. A bioreactor for the production of biomass, the bioreactor being configured to contain at least 15 liters of liquid comprising:
an external support structure displaying a general annular shape extending along a revolution axis X, and
a generally cylindrical shaped internal tank made of flexible material, said internal tank being configured to be secured inside and supported by the external support structure, the internal tank further displaying a closed extremity,
wherein the internal space of the internal tank is free of any internal structure,
wherein the bioreactor further comprises a sparger located at the closed extremity of the internal tank,
wherein the sparger is configured to generate bubbles displaying a diameter of at least 5 millimeters, and
wherein the sparger and the internal tank are arranged in a way that the generated bubbles form one bubble column once the internal tank is filled with liquid and the sparger is fed with gas, said bubble column enabling the liquid to be put in motion.

17. The bioreactor according to claim 16, wherein the sparger is arranged and secured in the center of the closed extremity, centered with regard to the revolution axis X.

18. The bioreactor according to claim 16, wherein the sparger comprises at least one radial aperture located along a revolution axis A of the sparger, said aperture being designed to generate a column of bubbles.

19. The bioreactor according to claim 18, wherein the sparger comprises two alignments of at least one radial aperture located along a revolution axis A of the sparger, the two alignments being on opposite sides of the sparger, said apertures being designed to generate at least one column of bubbles.

20. The bioreactor according to claim 16, wherein the diameter D of the generated bubbles ranges from 5 to 17 mm.

21. The bioreactor according to claim 16, wherein the bioreactor is configured to contain at least 100 liters of liquid.

22. The bioreactor according to claim 16, wherein the bioreactor is configured to contain at least 350 liters of liquid.

23. The bioreactor according to claim 16, wherein the bioreactor is configured to contain at least 1000 liters of liquid.

24. The bioreactor according to claim 16, wherein the internal tank is disposable.

25. The bioreactor according to claim 16, wherein it further comprises a control system.

26. The bioreactor according to claim 16, wherein the closed extremity displays a conical shape centered around the revolution axis X.

27. The bioreactor according to claim 26, wherein the sparger is designed to cooperate with the conical shape of the closed extremity.

28. The bioreactor according to claim 16, wherein the bioreactor is an air-lift system in which the liquid is solely put in motion by the generated air bubbles.

29. A system for the production of biomass, said system comprising the bioreactor according to claim 16 and a gas injection device, the gas injection device being put in fluidic communication with the sparger.

30. A method for growing biomass in a culture medium, said culture medium being put in motion by the bioreactor according to claim 16, said method comprising the following steps:
filling the internal tank with culture medium,
inserting the biomass aimed at being grown in the culture medium,
feeding the sparger with gas in order to generate the bubble column aimed at putting the culture medium into motion.
